(19) United States
(12) Patent Application Publication
(10) Pub. No.: US 2021/0293766 A1
(43) Pub. Date: Sep. 23, 2021

(54) PROCESS FOR ADJUSTING A GAS SUPPLY SYSTEM AND GAS SUPPLY SYSTEM WITH ADJUSTMENT FUNCTION

(71) Applicant: Dräger Safety AG & Co. KGaA, Lübeck (DE)

(72) Inventor: Tim GNOERRLICH, Lübeck (DE)

(21) Appl. No.: 17/203,142

(22) Filed: Mar. 16, 2021

(30) Foreign Application Priority Data
Mar. 17, 2020 (DE) 10 2020 001756.8

(51) Int. Cl. G01N 33/00

(52) U.S. Cl. CPC G01N 33/0006

(57) ABSTRACT

A process (100) for adjusting a gas supply system (300, 400) includes a calibration step for calibrating (101) a gas sensor (303, 401) at a first calibration time (t1) with a defined concentration of test gas. A first calibration result (S1) is determined during the calibration. At least one gas supply measured value (M1) is determined at a time (t2) preceding the first calibration time (t1). A determination step determines (103) a gas supply reference value (G1) by the at least one gas supply measured value (M1) being put into a mathematical relationship with the first calibration result (S1) and with a calibration result (S0) of the gas sensor (303, 401), which was determined at a time preceding the first calibration time (t1). An adjustment step adjusts (105) the gas supply system on the basis of the gas supply reference value (G1).

### CROSS REFERENCE TO RELATED APPLICATIONS

This application claims the benefit of priority under 35 U.S.C. § 119 of German Application 10 2020 001756.8, filed Mar. 17, 2020, the entire contents of which are incorporated herein by reference.

### TECHNICAL FIELD

The present invention pertains to a process for adjusting a gas supply system and to a gas supply system with adjustment function.

### TECHNICAL BACKGROUND

Electrochemical and catalytic sensors for monitoring toxic or explosive gases and vapors are commonly used. It is known that sensors, especially electrochemical sensors, change their measurement properties, especially their sensitivity, i.e., their sensitivity and/or their response time, in the course of their lifetime, until a measured value reaches 90% of an actually occurring gas concentration, and they must therefore be checked, especially calibrated regularly.

A calibration or calibrating is carried out, as a rule, in a two-step process, in which a zero point is determined in a first step by admitting a so-called “zero gas,” i.e., a gas that contains no impurities. The sensitivity of a respective sensor is determined in a second step by admitting a test gas, i.e., a gas with a defined concentration of impurities.

To generate a test gas, a gas generator may be used, which produces the test gas at regular intervals in order to test or to calibrate a respective sensor, for example, automatically. As an alternative, a test gas may be provided with a gas cylinder.

In case of a calibration without a gas generator, an operator or a user must carry out the calibration manually on site on a respective sensor by means of a gas cylinder, which is complicated, on the one hand, and may possibly be hazardous for the operator in a corresponding atmosphere, on the other hand.

In order to avoid incorrect interpretation of measured values determined by a sensor, a respective measuring system can be adjusted by adjusting or correcting a zero point and the sensitivity of the sensor as a function of a deviation detected during a testing or a calibration between a current state and a reference state of the sensor.

If a gas generator is used for the calibration or adjustment, this is also subject to an aging process and it generates an increasingly smaller quantity of gas over the lifetime at equal stimulus, which is interpreted by an automatic system for calibrating and adjusting a sensor as a drop in a response characteristic of the sensor, so that situations may occur in which the sensor is needlessly replaced or is incorrectly reported as being incorrect. The sensitivity of the sensor is correspondingly underestimated.

### SUMMARY

Against the above-described technical background, a basic object of the present invention is to provide a gas supply system, which is at least partly free from the above-described drawbacks. In particular, one object of the present invention is to maximize a time range between two calibration operations for calibrating a sensor of a gas measuring device.

The above object is accomplished by a process and by a gas supply system having the features of the invention. Further features and details of the present invention appear from the description and from the drawings. Features and details that are described in connection with the process according to the present invention are also valid in connection with the gas supply system according to the present invention and vice versa, so that reference is and can always mutually be made to the individual aspects of the present invention concerning the disclosure.

Thus, a process for adjusting a gas supply system is presented. The process comprises a calibration step for calibrating a gas sensor at a first calibration time with a defined concentration of test gas, wherein a first calibration result is determined during the calibration, and wherein at least one gas supply measured value determined at a time preceding the first calibration time is determined; a determination step for determining a gas supply reference value by putting the at least one gas supply measured value into a mathematical relationship with the first calibration result and with an additional calibration result of the gas sensor, which was determined at a time preceding the first calibration time; and an adjustment step for adjusting the gas supply system on the basis of the gas supply reference value.

A calibrating or a calibration is defined in the context of the invention being presented as an operation in which a current state of a gas sensor and/or of a gas supply of the gas measuring device being presented relative to a reference state is determined.

An adjustment or an adjusting is defined in the context of the present invention as an operation in which the gas sensor and/or the gas supply of the gas supply system being presented are adjusted or controlled, i.e., controlled or regulated as a function of a currently determined state of the gas sensor. An adjustment is defined, in particular, as an operation in which results of a calibration for correcting the gas supply system concerning a deviation between a current state and a reference state are used. For example, a respective measured value of a response characteristic of a respective gas sensor can be adjusted or corrected during an adjustment.

A computing unit is defined in the context of the invention being presented as a programmable processor unit or subprocessor unit. A computing unit may be configured as a computer, especially as a distributed computing system and it may be in a communicative contact with a memory, for example, a network memory or with a solid-state drive. In particular, a computing unit may be a control device, e.g., a microcontroller.

A defined concentration of test gas is defined in the context of the present invention as a predefined or known concentration of test gas, which is provided, for example, manually from a test gas cylinder or by means of a gas generator during a calibration operation. The defined concentration of test gas may fluctuate around a tolerance value.

The process being presented is used especially to adjust a gas supply, especially a gas generator, which is used to calibrate or to adjust a sensor. Provisions are made for this purpose for a gas supply to be also adjusted during a calibration or adjustment of a respective sensor.

An incorrect testing of a sensor, caused, e.g., by an incorrect actuation of the gas supply can be avoided by an adjusted gas supply. A time range between two calibration operations of a sensor can correspondingly be selected and correspondingly maximized independently from a state of aging of the gas supply.

The process being presented is based on the fact that a current or first calibration operation of a sensor is used to infer a state of aging of a gas supply, which admits test gas to the sensor. A first calibration result of the sensor, i.e., a sensitivity of the sensor due to an admission of test gas, is determined for this purpose during the first calibration operation, i.e., at a first calibration time.

In addition to the first calibration result, at least one gas supply measured value is determined at a time, which was detected by the sensor chronologically before, especially chronologically immediately before the first time at which calibration was performed by the sensor. In conjunction with an additional calibration result, which was determined, for example, before the first calibration time, a gas supply reference value can be determined on the basis of the gas supply measured value and the first calibration result. This means that a change in the sensitivity of the sensor between two calibration operations is put mathematically into a relationship with a gas supply measured value determined prior to the first calibration operation, especially with a last gas supply measured value prior to the calibration operation, in order to determine the gas supply reference value.

As soon as the gas supply reference value is known, this can be used to adjust a corresponding gas supply, i.e., to set it such that a quantity of test gas to be generated by the gas supply or an activation time of the gas supply is determined with the use of the gas supply reference value.

To determine the gas supply measured value, provisions may be made for a test gas to be admitted to a respective sensor regularly, especially between respective calibration operations.

The quantitative percentage of a deviation of the gas supply measured value from a desired value, which is due to the aging of the gas supply, can be determined by putting a change in the sensitivity of the sensor or an aging of the sensor into a mathematical relationship with a gas supply measured value. The quantitative percentage of the deviation of the gas supply measured value from the desired value, which is due to the aging of the gas supply, can be used, as soon as this quantitative percentage is known to adjust the gas supply, so that this percentage is compensated or is taken into consideration in further calibration operations.

Furthermore, a criterion of when the gas supply must be replaced because the quantity of gas generated by it and a corresponding signal-to-noise ratio are too low can be determined on the basis of the quantitative percentage of the deviation of the gas supply measured value from a desired value, i.e., the aging of the gas supply.

An aging of the gas sensor, i.e., a drop of measured values since a previous calibration or adjustment of the gas sensor, which drop is due to physical properties of the gas sensor, can be determined, in particular, by means of the process being presented after an adjustment of a gas sensor. This aging of the gas sensor can be compared to a result obtained for a drop in sensitivity of a gas supply. The relative aging of the gas generator corresponds in this case to a ratio of a currently determined reference value and an old reference value that was valid prior to the currently determined reference value. Correspondingly, the gas generator is not aged if reference values determined at different times are constant and equal.

The putting into a mathematical relationship, which is intended according to the present invention, may comprise a determination of a drop characteristic, e.g., an increase in the number of gas supply measured values. A determined drop characteristic can correspondingly be corrected during the adjustment operation according to the present invention. For example, a corresponding gas supply reference value can be set for this purpose at the gas supply system and/or a response characteristic of a respective gas sensor can be adjusted or corrected.

Provisions may be made for the calibration of the gas sensor and for the determination of the gas supply reference value to be repeated at a later time relative to the first calibration time, and the first calibration result is then used as an additional calibration result, for a calibration result determined at the later time to be used as the first calibration result, and for at least one gas supply measured value determined at a time preceding the later time to be used as at least one gas supply measured value.

Due to a repeated performance of respective partial steps of the process being presented, an aging process of a respective gas supply can be monitored continuously and correspondingly corrected. An influence of the aging process of the gas supply on a calibration operation can therefore be minimized and a respective sensor can be adjusted independently from a state of aging of the gas supply.

Furthermore, provisions may be made for the gas supply reference value to be determined by multiplying the at least one gas supply measured value by a ratio of the additional calibration result to the first calibration result.

The calculation instruction (1) for determining the gas supply reference value proved to be especially advantageous, and the following applies: S1 corresponds to a first calibration result, S0 to an additional calibration result, M1 to a gas supply measured value and G1 to a gas supply reference value. M1 may be, for example, a mean value or a median of a number N of last gas supply measured values prior to a first calibration operation for determining the first calibration result S1.

G1=M1*(S0/S1) (1)

Provisions may, furthermore, be made for the at least one gas supply measured value (M1) to be determined by one of the following determination steps:

a) Determination of an integral of a sensor response of the gas sensor (303, 401),
b) determination of a slope of a sensor response of the gas sensor (303, 401),
c) determination of a response time of a sensor response of the gas sensor (303, 401), and
d) determination of a curve of a sensor response of the gas sensor (303, 401).
Provisions may, furthermore, be made for the gas supply reference value to be compared to a predefined criterion in order to determine whether the gas supply must be replaced.

Provisions may be made, in particular, for a warning message to replace the gas supply to be outputted when a difference between the gas supply measured value and the gas supply reference value differs from a predefined threshold value.

Since a difference between the gas supply measured value and the gas supply reference value indicates a state of aging of the gas supply, the difference can be used as an indicator of the replacement of the gas supply. Thus, should, for example, the difference become greater than a predefined threshold value, it can be assumed that the gas supply is old and worn and is no longer suitable for calibrating a sensor.

Furthermore, provisions can be made for a value of the concentration of test gas, which is made available by means of the gas supply, to be corrected to the gas supply reference value, i.e., for example, the concentration is added up to the gas supply reference value, or the gas supply is controlled with the use of the gas supply reference value such that the concentration of test gas, which is provided by means of the gas supply, corresponds to a predefined desired value.

A gas supply can be controlled, i.e., controlled or regulated by means of the gas supply reference value calculated according to the present invention such that aging-related changes in the gas supply are compensated.

To adjust the gas supply system provided according to the present invention, an expected test gas curve shape of the gas supply system can be calibrated and adjusted. If, for example, a state of clogging of a sensor input is detected, for example, on the basis of the shape of a test gas curve but the shape of the test gas curve does change over time, the shape of this reference curve can also be adjusted at the time of a calibration of the sensor.

Furthermore, a gas supply signal, for example, a so-called “time to peak of the test gas impulse,” can also be calibrated and/or adjusted by means of the process being presented to determine a response time or a so-called “T90 time” of a respective sensor.

In a second aspect, the invention being presented pertains to a gas supply system with adjustment function. The gas supply system comprises an interface to a gas sensor, a gas supply and a computing unit. The computing unit is configured to determine a first calibration result at a first calibration time to calibrate the gas sensor with a defined concentration of test gas and to determine at least one gas supply measured value at a time preceding the first calibration time. Furthermore, the computing unit is configured to determine a gas supply reference value by the at least one gas supply measured value being put into a mathematical relationship with the first calibration result and with an additional calibration result for the gas sensor, which calibration result was determined at a time preceding the first time, and to adjust the gas supply system on the basis of the gas supply reference value.

The process being presented is used especially to operate the gas supply system being presented.

The gas supply system being presented comprises an interface to a gas sensor, so that the computing unit of the gas supply system is in communicative contact with the gas sensor and can receive, for example, measured values determined by the gas sensor. The interface may have a wired or wireless configuration. The gas sensor may correspondingly be configured as an integral component of the gas supply system or it may be connected as an external module to the gas supply system via the interface only. This means that the gas supply system may be configured to adjust itself or to adjust an external gas sensor.

Further measures improving the present invention appear from the following description of some exemplary embodiments of the present invention, which are shown in the figures. All the features and/or advantages appearing from the claims, from the description or from the drawings, including design details and arrangements in space, may be essential for the present invention both in themselves and in the different combinations. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic process diagram of a possible embodiment of the process being presented;

FIG. 2 is a diagram with measured values for carrying out the process being presented;

FIG. 3 is a schematic view of an embodiment of the gas supply system being presented with an integral gas sensor; and

FIG. 4 is a schematic view of another embodiment of the gas supply system being presented with an external gas sensor.

### DESCRIPTION OF PREFERRED EMBODIMENTS

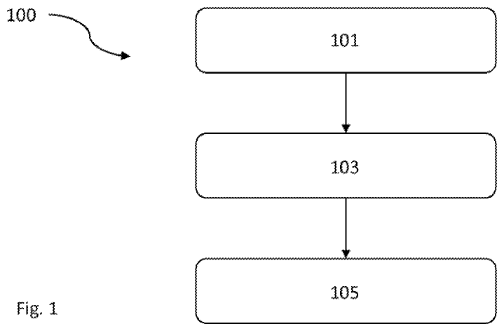

Referring to the drawings, Elements having the same function and mode of operation are provided with the same reference numbers in FIGS. 1 through 3.

FIG. 1 shows a process 100. The process 100 comprises a calibration step 101 for calibrating a gas sensor at a first calibration time with a defined concentration of test gas, wherein a first calibration result is determined during the calibration, and wherein at least one gas supply measured value determined at a time preceding the first calibration time is determined, a determination step 103 for determining a gas supply reference value by the at least one gas supply measured value being put into a mathematical relationship with the first calibration result and with an additional calibration result of the gas sensor, which was determined at a time preceding the first calibration time, and an adjustment step 105 for adjusting the gas supply system on the basis of the gas supply reference value.

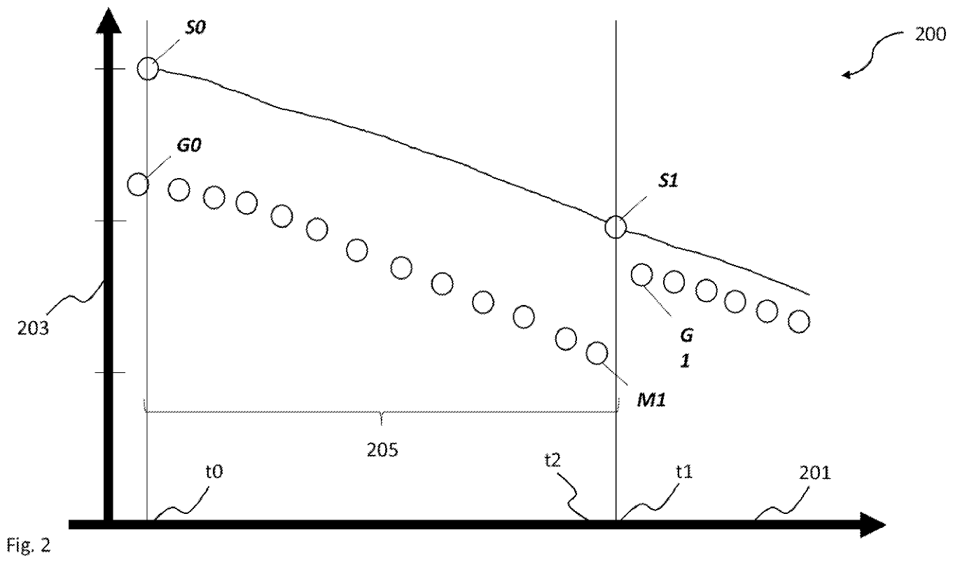

FIG. 2 shows a diagram 200. The diagram 200 extends over its abscissa 201 over time and over its ordinate 203 over a sensor sensitivity in μA/ppm.

A gas sensor of a gas supply system is calibrated at a first calibration time t1 by, for example, an operator with a test gas cylinder, which contains a defined concentration of target gas or test gas.

The test particles discharged from the test gas cylinder migrate to a gas sensor, so that the gas sensor measures a gas supply measured value with a sensor sensitivity of, e.g., 1.5 μA/ppm, which corresponds to a first calibration result S1.

A basic gas supply reference value G0 of, e.g., 2000 ppm·sec, which was determined or preset at a time t0 prior to the first calibration time t1, is set at the gas supply system during the first calibration time t1. Dropping gas generator signals 205 are seen over the use time of the gas sensor and of the gas generator, so that a gas supply measured value (M1), which is determined at a time (t2) preceding the first calibration time (t1), corresponds, for example, to 1000 ppm·sec.

Starting from the gas supply measured value (M1), the gas supply system would assign a sensitivity of M1/G0=1 μA/ppm to the gas sensor, because the drop of the gas generator signals 205 would be fully assigned to the gas sensor.

However, the gas generator has, indeed, aged as well, so that an operator calibration at the first calibration time t1 yields a “true” sensor sensitivity of S1=1.5 μA/ppm.

This means that the gas sensor and the gas generator are aged at the same ratio. Based on this knowledge, a new gas supply reference value G1 can be determined by means of the calculation instruction (1), where S1 corresponds to a first calibration result, S0 to an additional calibration result, M1 to a gas supply measured value and G1 to a gas supply reference value. M1 may be a mean value or median of a number N of last gas generator signals prior to a first calibration operation to determine the first calibration result S1.

G1=M1*(S0/S1) (1)

The process 100 may be used, for example, with sensor raw values, of, e.g., currents of an electrochemical sensor or with already calculated concentration values of a sensor.

A “continuous admission” of gas can be carried out to determine the sensitivity of the gas sensor during a calibration. A stable measured value is then expected and the sensitivity is determined. For example, an integral of a sensor response of the gas response to an admission of a test gas, a jump of a sensor response of the gas sensor in response to the admission of a test gas or a time until the peak of the sensor response of the gas sensor in response to the admission of a test gas can be used to determine the sensitivity of the gas sensor during a calibration.

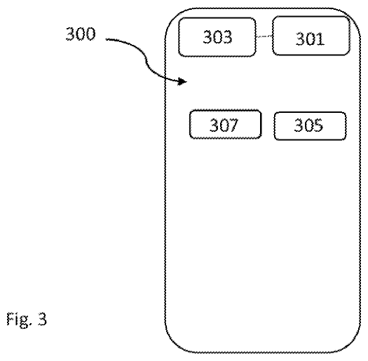

FIG. 3 shows a gas supply system 300. The gas supply system 300 comprises an interface 301 to a gas sensor, a gas sensor 303, a gas supply 305 and a computing unit 307. The computing unit is configured to determine a first calibration result at a first calibration time to calibrate the gas sensor with a defined concentration of test gas.

Furthermore, the computing unit 307 is configured to determine at least one gas supply measured value at a time preceding the first calibration time.

Furthermore, the computing unit 307 is configured to determine a gas supply reference value by the at least one gas supply measured value being put into a mathematical relationship with the first calibration result and with an additional calibration result of the gas sensor, which was determined at a time preceding the first calibration time.

Furthermore, the computing unit 307 is configured to adjust the gas supply system 300 on the basis of the gas supply reference value.

In the example shown in FIG. 3, the gas sensor 303 is connected to the interface 301 in a communicative manner via an electrical connection, for example, a cable or a soldered connection.

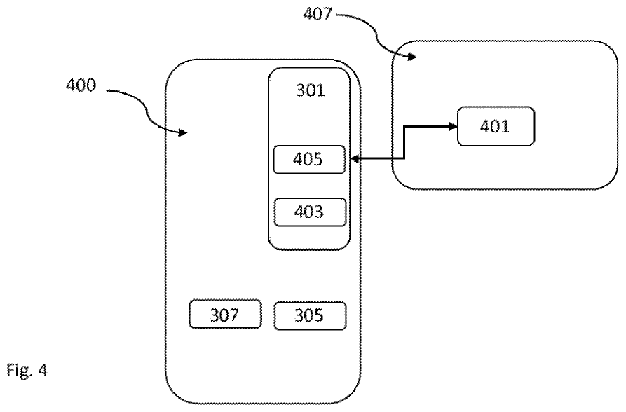

FIG. 4 shows a gas supply system 400. The gas supply system 400 is identical to the gas supply system 300 according to FIG. 3 with the exception that the interface 301 is connected to an external gas sensor 401 in, for example, a gas measuring device 407 via a wireless interface. The gas supply system 400 correspondingly forms a portable module for testing different gas sensors.

The interface 301 may comprise a first communication module 403 for wireless communication via, e.g., WLAN, Bluetooth, Nearfield Communication, Zigbee or a mobile wireless device signal, e.g., 5G, and a second communication module 405 for connecting a cable, e.g., a COM port or a USB port.

The gas supply system 400 may optionally comprise an output unit 409, e.g., an LED, a speaker and/or a display, in order for warning messages, e.g., an instruction to replace the gas sensor 401, to be outputted.

While specific embodiments of the invention have been shown and described in detail to illustrate the application of the principles of the invention, it will be understood that the invention may be embodied otherwise without departing from such principles.

### LIST OF REFERENCE NUMBERS

100 Process
101 Calibration step
103 Determination step
105 Adjustment step
200 Diagram
201 Abscissa
203 Ordinate
205 Gas generator signals
t1 First calibration time
S1 First calibration result
M1 Gas supply measured value
t2 Preceding time
G0 Gas supply reference value
G1 New gas supply reference value
300 Gas supply system
301 Interface
303 Gas sensor
305 Gas supply
307 Computing unit
400 Gas supply system
401 External gas sensor
403 First communication module
405 Second communication nodule
407 Gas measuring device
409 Output unit

What is claimed is:

1. A process for adjusting a gas supply system, the process comprising the steps of:
calibrating a gas sensor at a first calibration time with a defined concentration of test gas, wherein a first calibration result is determined during the calibration, and at least one gas supply measured value is determined at a time preceding the first calibration time;
determining a gas supply reference value by putting at least one gas supply measured value into a mathematical relationship with the first calibration result and with a calibration result of the gas sensor, which was determined at another time preceding the first calibration time; and
adjusting the gas supply system based on the gas supply reference value.

2. A process in accordance with claim 1, wherein:
the calibration of the gas sensor and the determination of the gas supply reference value are repeated at a later time compared to the first calibration time; and
the first calibration result is then used as an additional calibration result, a calibration result determined at the later time is used as the first calibration result, and at least one gas supply measured value determined at a time preceding the later time is used as at least one gas supply measured value.

3. A process in accordance with claim 2, wherein the gas supply reference value is determined by the at least one gas supply measured value being multiplied by a ratio of the additional calibration result to the first calibration result.

4. A process in accordance with claim 1, wherein the at least one gas supply measured value is determined by one of the following determination steps:
determining an integral of a sensor response of the gas sensor;
determining a slope of a sensor response of the gas sensor;
determining a response time of a sensor response of the gas sensor; and
determining a curve shape of a sensor response of the gas sensor.

5. A process in accordance with claim 1, wherein a warning message is outputted depending on a comparison of the gas supply reference value with a predefined criterion.

6. A process in accordance with claim 5, wherein the warning message for replacing a gas supply of the gas supply system is outputted when a difference between the gas supply measured value and the gas supply reference value differs from a predefined threshold value.

7. A process in accordance with claim 1, wherein a value of the concentration of test gas, which concentration is provided by means of the gas supply, is corrected to the gas supply reference value or the gas supply is controlled with the use of the gas supply reference value such that the concentration of test gas, which concentration is provided by means of the gas supply, corresponds to a predefined threshold value.

8. A gas supply system with adjustment function, the gas supply system comprising:
an interface to a gas sensor;
a gas supply; and
a computing unit configured to determine a first calibration result at a first calibration time to calibrate the gas sensor connected to the interface with a defined concentration of test gas to determine at least one gas supply measured value at a time preceding the first calibration time, and to determine a gas supply reference value by the at least one gas supply measured value being put into a mathematical relationship with the first calibration result and with an additional calibration result of the gas sensor, which additional calibration result was determined at a time preceding the first calibration time, and to adjust the gas supply system based on the gas supply reference value.

9. A gas supply system in accordance with claim 8, wherein:
the calibration of the gas sensor and the determination of the gas supply reference value are repeated at a later time compared to the first calibration time; and
the first calibration result is then used as an additional calibration result, a calibration result determined at the later time is used as the first calibration result, and at least one gas supply measured value determined at a time preceding the later time is used as at least one gas supply measured value.

10. A gas supply system in accordance with claim 9, wherein the gas supply reference value is determined by the at least one gas supply measured value being multiplied by a ratio of the additional calibration result to the first calibration result.

11. A gas supply system in accordance with claim 8, wherein the computing unit is configured to determine the at least one gas supply measured value by one of the following determination steps:
determining an integral of a sensor response of the gas sensor;
determining a slope of a sensor response of the gas sensor;
determining a response time of a sensor response of the gas sensor; and
determining a curve shape of a sensor response of the gas sensor.

12. A gas supply system in accordance with claim 8, wherein a warning message is outputted depending on a comparison of the gas supply reference value with a predefined criterion.

13. A gas supply system in accordance with claim 12, wherein the warning message for replacing a gas supply of the gas supply system is outputted when a difference between the gas supply measured value and the gas supply reference value differs from a predefined threshold value.

14. A gas supply system in accordance with claim 8, wherein a value of the concentration of test gas, which concentration is provided by means of the gas supply, is corrected to the gas supply reference value or the gas supply is controlled with the use of the gas supply reference value such that the concentration of test gas, which concentration is provided by means of the gas supply, corresponds to a predefined threshold value.

15. A gas supply system in accordance with claim 8, wherein the gas supply comprises a gas generator or a gas cylinder.
